(19) United States
(12) Patent Application Publication
(10) Pub. No.: US 2023/0404485 A1
(43) Pub. Date: Dec. 21, 2023

(54) TERMINAL APPARATUS, INFORMATION ACQUISITION SYSTEM AND INFORMATION PROCESSING METHOD

(71) Applicant: Nippon Telegraph and Telephone Corporation, Tokyo (JP)

(72) Inventors: Kunio KASHINO, Musashino-shi, Tokyo (JP); Masahiro NAKANO, Musashino-shi, Tokyo (JP); Ryohei SHIBUE, Musashino-shi, Tokyo (JP)

(21) Appl. No.: 18/036,083

(22) PCT Filed: Nov. 12, 2020
(86) PCT No.: PCT/JP2020/042330

§ 371 Date: May 9, 2023

(51) Int. Cl. A61B 5/00; G16H 40/67

(52) U.S. Cl. CPC A61B 5/684; A61B 5/0015; G16H 40/67

(57) ABSTRACT

According to an aspect of the present invention, there is provided a terminal apparatus including an operator that receives information indicating a position in a body of a subject from a user, in which biological information corresponding to the position received by the operator is output on the basis of biological information obtained by a plurality of sensors of which positions are substantially fixedly maintained with respect to the body of the subject.

### TECHNICAL FIELD

The present invention relates to a technique for a terminal apparatus, an information acquisition system and an information processing method.

### BACKGROUND ART

In the related art, auscultation using a stethoscope has been widely used for acquisition of an acoustic signal from a living body. As specific examples of a stethoscope, there are a stethoscope in which a tube is connected to a chest piece to guide sound to ears, and an electronic stethoscope in which an acoustic sensor is applied to a chest piece to electrically guide sound to ears (for example, refer to Non Patent Literature 1).

### CITATION LIST

### Non Patent Literature

Non Patent Literature 1: Shuang Leng and five others, “The electronic stethoscope”, BioMedical Engineering Online (2015), DOI 10.1186/s12938-015-0056-y

### SUMMARY OF INVENTION

### Technical Problem

In auscultation of the related art, it is necessary for a medical practitioner to be located facing a patient and to apply a stethoscope to a desired site of the patient's body. Therefore, auscultation has been performed in a state in which the medical practitioner and the patient are close to each other. Such a problem is a problem common not only to an examination using a stethoscope but also to general work of acquiring information (biological information) in a body of an examination subject.

In view of the above circumstances, an object of the present invention is to provide a technique capable of acquiring biological information of a desired site in a body of a subject without approaching the subject.

### Solution to Problem

According to an aspect of the present invention, there is provided a terminal apparatus including an operator that receives information indicating a position in a body of a subject from a user, in which biological information corresponding to the position received by the operator is output on the basis of biological information obtained by a plurality of sensors of which positions are substantially fixedly maintained with respect to the body of the subject.

According to another aspect of the present invention, there is provided an information acquisition system including a holding tool; and a terminal apparatus, in which the holding tool includes a holding tool main body and a plurality of sensors of which positions are substantially fixedly maintained in the holding tool main body, the terminal apparatus includes an operator that receives information indicating a position in a body of a subject from a user, and the terminal apparatus outputs biological information corresponding to the position received by the operator on the basis of biological information obtained by a plurality of sensors of which positions are maintained substantially fixedly with respect to the body of the subject.

According to still another aspect of the present invention, there is provided an information processing method of causing a computer provided with an operator that receives information indicating a position in a body of a subject from a user to execute receiving the information indicating the position in accordance with an operation of the user on the operator; and outputting biological information corresponding to the position received by the operator on the basis of biological information obtained by a plurality of sensors of which positions are maintained substantially fixedly with respect to the body of the subject.

### Advantageous Effects of Invention

According to the present invention, it is possible to acquire biological information of a desired site in a body of a subject without approaching the subject.

### BRIEF DESCRIPTION OF DRAWINGS

FIG. 1 is a diagram illustrating a system configuration example of an information acquisition system 100 according to a first embodiment of the present invention.

FIG. 2 is a diagram illustrating a system configuration example of an information acquisition system 100 according to a second embodiment of the present invention.

FIG. 3 is a diagram illustrating a system configuration example of an information acquisition system 100 according to a third embodiment of the present invention.

FIG. 4 is a diagram illustrating a specific example of a holding tool 10 used in an information acquisition system 100 according to a fourth embodiment of the present invention.

FIG. 5 is a diagram illustrating a system configuration example of an information acquisition system 100 according to a fifth embodiment of the present invention.

FIG. 6 is a diagram illustrating a system configuration example of an information acquisition system 100 according to a sixth embodiment of the present invention.

FIG. 7 is a diagram illustrating a system configuration example of an information acquisition system 100 according to a seventh embodiment of the present invention.

FIG. 8 is a diagram illustrating a system configuration example of an information acquisition system 100 according to an eighth embodiment of the present invention.

FIG. 9 is a diagram illustrating a system configuration example of an information acquisition system 100 according to a ninth embodiment of the present invention.

FIG. 10 is a diagram illustrating a system configuration example of an information acquisition system 100 according to a tenth embodiment of the present invention.

FIG. 11 is a diagram illustrating a specific example of an operation screen used in an operator (202, 210) of a terminal apparatus 20 in the first to tenth embodiments.

### DESCRIPTION OF EMBODIMENTS

Embodiments of the present invention will be described in detail with reference to the drawings.

### First Embodiment

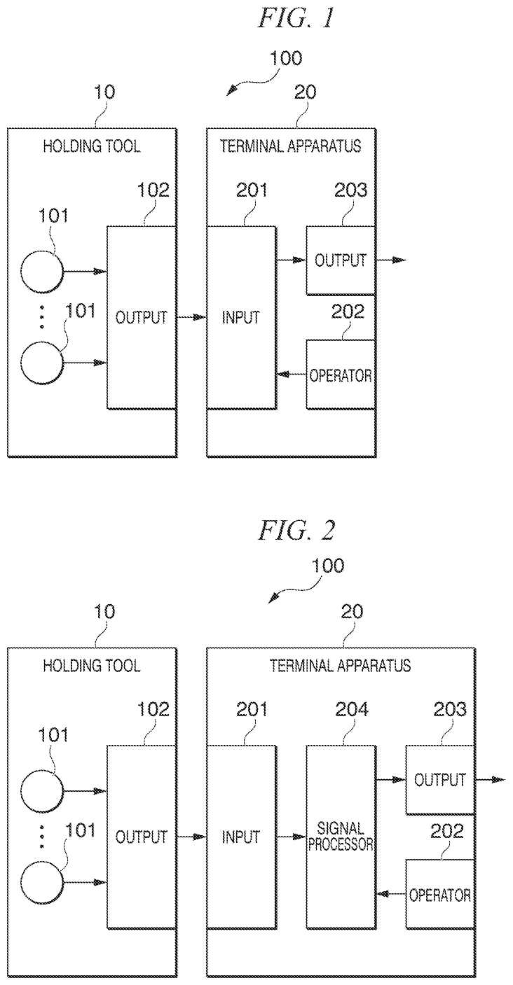

FIG. 1 is a diagram illustrating a system configuration example of an information acquisition system 100 according to a first embodiment of the present invention. The information acquisition system 100 according to the first embodiment includes a holding tool 10 and a terminal apparatus 20. The holding tool 10 includes a plurality of sensors 101 and an output 102. The holding tool 10 substantially fixedly maintains a relative distance between a body of a person of which information in the body is to be acquired (hereinafter, referred to as a “subject”) and each sensor 101.

For example, the holding tool 10 may be a apparatus worn on the body of the subject. Specifically, the holding tool 10 may be configured by using an article such as a helmet, sunglasses, glasses, a headband, a mask, a bracelet, a ring, a leg ring, a corset, or a shoe. The holding tool 10 may be a garment worn by the subject. Specifically, the holding tool 10 may be configured by using an article such as outerwear, an undergarment, a T-shirt, a jacket, a shirt, trousers, pants, a hat, a sock, a glove, or a belt. The holding tool 10 may be an article used by coming contact with a part of the body of the subject. Specifically, the holding tool 10 may be configured by using an article such as a bed, a sofa, a chair, a wall, or a specific plate (a plate against which the body is pressed by the subject). The holding tool 10 may be configured by using any article as long as the article can substantially fixedly maintain a relative distance between the body of the subject and each sensor 101.

A position of the sensor 101 is substantially fixed in the holding tool 10. As the sensor 101, any sensor may be used as long as it can obtain information regarding the body of the subject (hereinafter, referred to as “biological information”). For example, a sensor for measuring an electrocardiogram may be used as the sensor 101, a sensor (for example, a pressure sensor) for measuring blood pressure may be used, or a sensor (for example, an acoustic sensor) for measuring sound may be used. In a case where the electrocardiogram is measured, the biological information is information indicating a waveform of the electrocardiogram. In a case where the blood pressure is measured, the biological information is information indicating the blood pressure. In a case where sound is measured, the biological information is information indicating the sound.

For one holding tool 10, only one type of sensor 101 may be used, or a plurality of types of sensors 101 may be used. In the following description, an embodiment in which an acoustic sensor is applied as the sensor 101 will be described.

The sensor 101 outputs information (hereinafter, referred to as “sound information”) indicating sound generated in the surroundings to the output 102. For example, the sensor 101 may convert an acoustic signal generated in the surroundings into an electrical signal and output the electrical signal to the output 102. In this case, the electrical signal is a specific example of the sound information.

The output 102 outputs the sound information output from each sensor 101 to the terminal apparatus 20 such that the sensor 101 from which the sound information is obtained can be identified. The output 102 may output the sound information through, for example, wired communication using a cable, or may output the sound information through wireless communication. The output 102 may output the electrical signal output from the sensor 101 without particular processing, for example, without executing a modulation process. In this case, the output 102 may output the electrical signal by using a cable allocated to each sensor 101. The output 102 may output the sound information by using a wired communication protocol such as USB. The output 102 may output the sound information by using a wireless communication protocol such as Bluetooth (registered trademark) or infrared communication.

The terminal apparatus 20 may be configured by using a wearable terminal such as a smartphone, a tablet, or a smart watch, or a general-purpose information apparatus such as a game machine or a television receiver, or may be configured as a dedicated apparatus. The terminal apparatus 20 includes an input 201, an operator 202, and an output 203.

The input 201 acquires the sound information output from the output 102. The input 201 may have any configuration as long as the configuration enables acquisition of sound information in accordance with an output mode (for example, a communication protocol) used in a case where the output 102 outputs the sound information. The input 201 outputs sound information corresponding to an instruction from the operator 202 among pieces of acquired sound information. For example, in a case where the sensor 101 is directly designated by using the operator 202, the input 201 selects sound information output by the designated sensor 101 and outputs the sound information to the output 203. For example, in a case where a position such as a site of the body is designated by using the operator 202, the input 201 selects sound information output by the sensor 101 corresponding to the designated position and outputs the sound information to the output 203. The sensor 101 corresponding to the designated position may be, for example, the sensor 101 closest to the designated position, or may be one or a plurality of sensors 101 at a distance within a predetermined range from the designated position. In a case where a plurality of sensors 101 are selected, the input 201 may output statistical values (for example, average values) of the sound information, or may combine and output a plurality of pieces of sound information.

The operator 202 is configured by using an existing input apparatus such as a keyboard, a pointing apparatus (a mouse, a tablet, or the like), a button, or a touch panel. In a case where the operator 202 is configured by using a keyboard or a pointing apparatus, a display apparatus that displays an operation screen to be operated is also provided in the terminal apparatus 20.

When an instruction from a person (hereinafter, referred to as a “user”) who intends to acquire sound information is input to the terminal apparatus 20, the operator 202 is operated by the user. The operator 202 may be an interface for connecting the input apparatus to the terminal apparatus 20. In this case, the operator 202 inputs an input signal generated in response to a user's input in the input apparatus to the terminal apparatus 20. The operator 202 may be configured by using a microphone and a voice recognition apparatus. In this case, the operator 202 performs voice recognition of words said by the user, and inputs text information as a recognition result to the terminal apparatus 20. The operator 202 may be configured in any manner as long as a user's instruction can be input to the terminal apparatus 20. The user's instruction input on the operator 202 includes at least information indicating the position of the body of the subject directly or indirectly. For example, the user's instruction may be configured to indicate a name of a site of the body of the subject, may be configured to indicate a position in a model obtained by generalizing the body of the subject, or may be configured to designate one or a plurality of sensors 101.

The output 203 outputs the information output from the input 201 to another apparatus. The output 203 may output the sound information to another apparatus by using, for example, wired communication or wireless communication, or may record the sound information in a connected storage apparatus. The output 203 may be configured in any manner as long as it can output the sound information to the outside of the terminal apparatus 20.

In the information acquisition system 100 according to the first embodiment configured as described above, the user can acquire biological information at a desired position of the body of the subject by operating the operator 202 of the terminal apparatus 20. The biological information obtained at that time is information based on biological information obtained by the plurality of sensors 101 provided in the holding tool 10, and it is not necessary for the user to directly move the sensor 101 by using a hand or the like. Therefore, it is possible to acquire biological information of a desired site of the body of the subject without the user approaching the subject.

The information acquisition system 100 may be used in a situation in which a subject is a patient and a user is a medical practitioner in a medical field. In particular, by configuring the holding tool 10 to be mountable by the patient, the medical practitioner can designate and auscultate any biological information in each site of the patient (for example, sound of each site on a body surface) without approaching and facing the patient.

In the above description, the processing of selecting and outputting sound information according to an instruction from the operator 202 among pieces of acquired sound information is performed by the input 201, but the processing corresponding to such selection may be performed by the output 203 instead of the input 201. In this case, information indicating the operation content received by the operator 202 may be output to the output 203 instead of the input 201.

### Second Embodiment

FIG. 2 is a diagram illustrating a system configuration example of an information acquisition system 100 according to a second embodiment of the present invention. The information acquisition system 100 according to the second embodiment is different from the information acquisition system 100 according to the first embodiment in that the terminal apparatus 20 further includes a signal processor 204. According to the difference in the configuration, the input 201 may directly sound information obtained from the output 102 to the signal processor 204 without any processing.

The signal processor 204 performs signal processing by using a plurality of pieces of sound information input by the input 201, and outputs sound information obtained by the signal processing to the output 203. The signal processor 204 may be configured by using a processor such as a central processing unit (CPU) and a memory. In this case, the processor functions as the signal processor 204 by executing the program. All or some of functions of the signal processor 204 may be realized by using hardware such as an application specific integrated circuit (ASIC), a programmable logic apparatus (PLD), or a field programmable gate array (FPGA). The program may be recorded in a computer-readable recording medium. The computer-readable recording medium is, for example, a portable medium such as a flexible disk, a magneto-optical disc, a ROM, a CD-ROM, or a semiconductor storage apparatus (for example, a solid state drive (SSD)), or a storage apparatus such as a hard disk or a semiconductor storage apparatus built in a computer system. The program may be transmitted via an electric communication line.

The signal processor 204 outputs sound information to the output 203 according to a user's instruction input by the operator 202. For example, sound information from one or a plurality of sensors 101 estimated to be spatially closest to a body site indicated by the user's instruction input by the operator 202 may be selected, and the selected sound information may be output to the output 203 as main sound information. In this case, the signal processor 204 may have information indicating a position where each sensor 101 in the holding tool 10 is located with respect to the body of the subject. For example, in a case where a method in which the subject uses the holding tool 10 is determined in advance (for example, in a case where the holding tool is worn), a correspondence relationship between each sensor 101 and a position thereof on the body of the subject is determined in advance.

The processing of outputting sound information as main sound information is, for example, processing of outputting the selected sound information with a larger relative difference in signal intensity between the selected sound information and other sound information. For example, the signal processor 204 may amplify the main sound information, suppress other sound information, and output the main sound information to the output 203. For example, the signal processor 204 may simply output only the main sound information to the output 203. The signal processor 204 may perform signal processing in any aspect as long as the main sound information is acquired to be able to be used by the user. The use by the user may be, for example, medical diagnosis or analysis.

For example, the signal processor 204 may form a beam through beamforming according to a position of a site of the body indicated by the user's instruction input by the operator 202, and generate sound information having directivity. More specifically, the signal processing may be performed such that sound information arriving from a direction indicating the site of the body indicated by the user's instruction is a stronger signal than sound information arriving from other directions. The signal processing may be performed such that not only sound information generated in the direction but also in a specific region in the direction is a signal stronger than the sound information generated in other regions.

The signal processor 204 may generate sound information to be output by performing sound field estimation. Specifically, the reason is as follows. The signal processor 204 estimates sound information acquired by the sensor 101 in a case where the sensor 101 is assumed to be disposed in a region including a position of the body indicated by the user's instruction. This processing may be performed, for example, by estimating a sound source and then multiplying the sound source by a transfer function from each sound source to a location designated by the user to estimate sound information at the designated location. The signal processor 204 outputs the estimated sound information to the output 203.

In the information acquisition system 100 according to the second embodiment configured as described above, the user can acquire biological information at a desired position of the body of the subject by operating the operator 202 of the terminal apparatus 20. In particular, in a case where processing of forming a beam through beamforming is performed by the signal processor 204, sound information generated in a specific direction or a specific region can be acquired.

For example, it is also possible to estimate and listen to the sound in the body of the subject by performing the above-described signal processing. For example, in the heart, attention is paid to a valve inside the heart, attention is paid to heart noise generated near the valve, and a degree and a mode of the noise can be heard. Such an effect is an excellent effect that is difficult to realize by the stethoscope of the related art.

### Third Embodiment

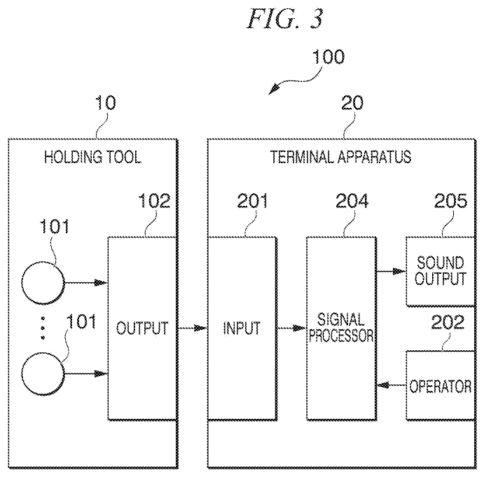

FIG. 3 is a diagram illustrating a system configuration example of an information acquisition system 100 according to a third embodiment of the present invention. The information acquisition system 100 according to the third embodiment is different from the information acquisition system 100 according to the second embodiment in that the terminal apparatus 20 further includes a sound output 205 instead of the output 203. The sound output 205 is an example of a specific aspect of the output 203. According to this difference in configuration, the signal processor 204 outputs sound information to the sound output 205 instead of the output 203.

The sound output 205 outputs sound information such that a user can listen to the sound information output by the signal processor 204. For example, the sound output 205 may be configured as a speaker built in the terminal apparatus 20. For example, the sound output 205 may be configured as an output interface that outputs a sound signal to a speaker (including a headphone or an earphone) as an external apparatus connected to the terminal apparatus 20.

In the information acquisition system 100 according to the third embodiment configured as described above, the user can listen to sound of a desired site of the subject from the terminal apparatus 20 via the sound output 205.

### Fourth Embodiment

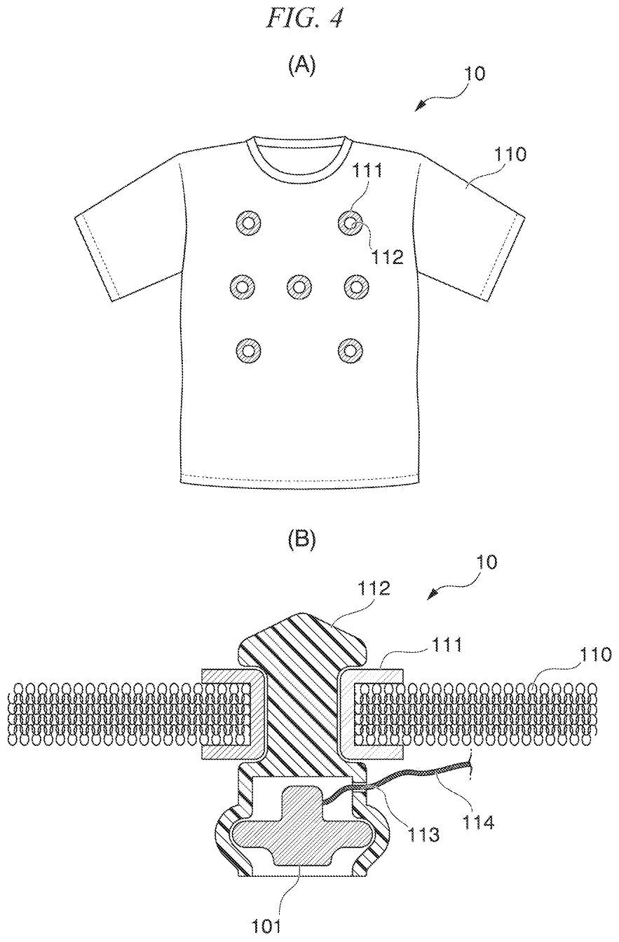

FIG. 4 is a diagram illustrating a specific example of a holding tool 10 used in an information acquisition system 100 according to a fourth embodiment of the present invention. The information acquisition system 100 according to the fourth embodiment is the same as the information acquisition system 100 according to the third embodiment except for the configuration of the holding tool 10. A specific example of a configuration of the holding tool 10 described below is not necessarily limited to being used as the third embodiment and the fourth embodiment, and can also be applied to the first embodiment and the second embodiment, and can be applied to a fifth embodiment to an eleventh embodiment that will be described later.

In FIG. 4, the holding tool 10 is configured by using an article that can be worn by a subject. In particular, in FIG. 4, the holding tool 10 is configured as clothing (more specifically, a T-shirt). A plurality of sensor fixtures 111 are provided on clothing fabric 110, and a housing 112 of a sensor 101 is attached to each sensor fixture 111. The sensor fixture 111 may be configured by using, for example, a grommet. The housing 112 is fitted into the grommet to be substantially fixed at a predetermined position of the clothing fabric 110. The sensor 101 is attached to a part of the housing 112. A hole penetrating from the inside to the outside is provided in a part of the housing 112, and a cord 114 of the sensor 101 is connected to the output 102 through the hole. Power may be supplied to the sensor 101 via the cord 114.

By configuring the holding tool 10 as clothing as described above, the plurality of sensors 101 can be easily disposed at predetermined positions on the body of the subject. Since the sensor 101 is disposed at an appropriate location only by the subject wearing the holding tool 10 configured as clothing, there is no need for a person having detailed knowledge (for example, a medical practitioner or a user) to give an instruction or the like to the subject.

### Fifth Embodiment

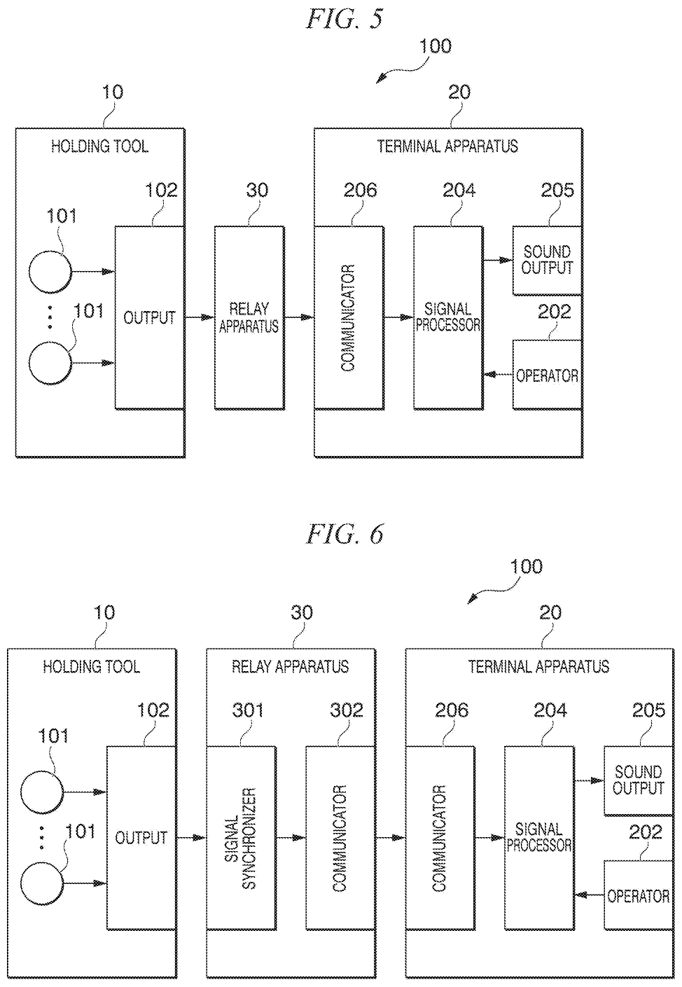

FIG. 5 is a diagram illustrating a system configuration example of an information acquisition system 100 according to a fifth embodiment of the present invention. The information acquisition system 100 according to the fifth embodiment is different from the information acquisition system 100 according to the third embodiment in that the information acquisition system 100 further includes a relay apparatus 30 and the terminal apparatus 20 includes a communicator 206 instead of the input 201. According to the difference in the configuration, the output 102 outputs a sound signal to the relay apparatus 30. The signal processor 204 acquires the sound information from the communicator 206.

The relay apparatus 30 is communicatively connected to the output 102 of the holding tool 10. In this respect, a configuration between the relay apparatus 30 and the output 102 may be the same as the configuration between the input 201 and the output 102 in the first to third embodiments. The relay apparatus 30 performs data communication with the terminal apparatus 20. Data communication performed between the relay apparatus 30 and the terminal apparatus 20 may be performed via, for example, a data communication network such as a local area network (LAN) or the Internet. The relay apparatus 30 may digitize a sound signal output from the sensor 101 as linear PCM at a sampling frequency of 48 kHz and a quantization accuracy of 16 bits, and transmit the digitized signal by using an Internet protocol.

In the information acquisition system 100 according to the fifth embodiment configured as described above, the user can acquire biological information of a desired site of the body of the subject even if the subject and the user are separated from each other at a longer distance.

### Sixth Embodiment

FIG. 6 is a diagram illustrating a system configuration example of an information acquisition system 100 according to a sixth embodiment of the present invention. The information acquisition system 100 according to the sixth embodiment is different from the information acquisition system 100 according to the fifth embodiment in that the relay apparatus 30 includes a signal synchronizer 301 and a communicator 302.

The signal synchronizer 301 acquires sound information output from the output 102 of the holding tool 10. A configuration between the signal synchronizer 301 and the output 102 may be the same as the configuration between the input 201 and the output 102 in the first to third embodiments. The signal synchronizer 301 secures synchronization of signals obtained from the sensor 101.

In a case where the number of sensors 101 is large, there is a possibility of employing a configuration in which signals output from the sensors 101 are separately acquired by a plurality of subsystems. In a case where such a configuration is employed, subsystems may be driven by individual clocks. With such a configuration, it may be difficult to secure strict synchronization in all the sensors 101. In a case where synchronization is not secured, subsequent signal processing may be hindered.

The signal synchronizer 301 can solve such a problem. For example, the signal synchronizer 301 may periodically output the same synchronization signal to all the subsystems in common. The respective subsystems perform synchronization on the basis of the synchronization signal output from the signal synchronizer 301. The synchronization signal may be output from the signal synchronizer 301 in a frequency band or a time section that does not interfere with observation of a target signal (for example, a signal regarding sound information).

A method may be employed in which a plurality of subsystems have flags that can be referred to by each other to synchronize an acquired signal regarding sound information with real time. Such a configuration can also be applied to a case where the plurality of sensors 101 target different types of signals (for example, in a case where some sensors 101 output sound signals and other sensors 101 output electrocardiographic signals).

In the information acquisition system 100 according to the sixth embodiment configured as described above, even in a case where the number of sensors 101 provided in the holding tool 10 is large, the accuracy of acquired information can be enhanced.

### Seventh Embodiment

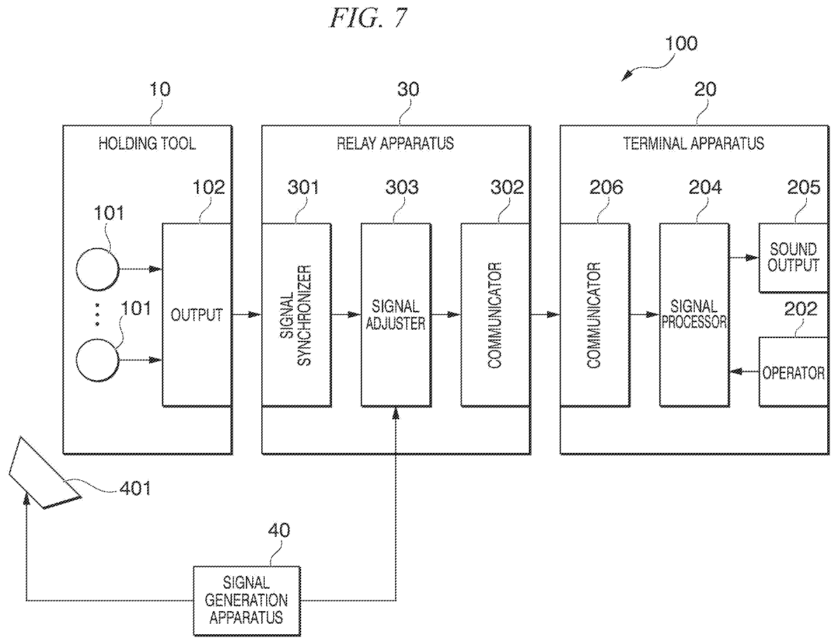

FIG. 7 is a diagram illustrating a system configuration example of an information acquisition system 100 according to a seventh embodiment of the present invention. The information acquisition system 100 according to the seventh embodiment is different from the information acquisition system 100 according to the sixth embodiment in that the information acquisition system 100 further includes a signal generation apparatus 40 and in that the relay apparatus 30 further includes a signal adjuster 303.

The signal generation apparatus 40 includes a signal output 401. The signal generation apparatus 40 generates a predetermined signal (known signal), and outputs the signal from the signal output 401 in a mode perceivable by the sensor 101. For example, in a case where the sensor 101 is an acoustic sensor, the signal output 401 is configured by using a apparatus such as a speaker that generates sound. The signal output from the signal output 401 is acquired by the sensor 101, and the sensor 101 outputs information (sound information) corresponding to a predetermined signal. Specific examples of the predetermined signal include signals such as an impulse signal, a chirp signal, and band noise.

The signal adjuster 303 compares the received signal with the signal generated in the signal generation apparatus 40 to measure a signal transmission time from a signal generation source (signal output 401) to each sensor 101. The signal adjuster 303 estimates a position of each sensor 101 on the basis of the measurement result. The signal adjuster 303 determines whether or not the position of each sensor 101 is maintained within a normal range on the basis of the position estimation result.

The signal adjuster 303 applies a filter having inverse characteristics of measured frequency characteristics to an output signal from each sensor 101. Through such processing, transmission characteristics can be compensated. A plurality of signal outputs 401 may be provided. The signal output 401 may be provided in the holding tool 10.

In the information acquisition system 100 according to the sixth embodiment configured as described above, in a case where a deviation occurs in a position of the sensor 101, the positional deviation can be detected. For example, in a case where the signal adjuster 303 includes a apparatus that outputs sound, light, an image, a character, vibration, or the like, the signal adjuster 303 may output sound, light, an image, a character, vibration, or the like in a case where the sensor 101 that is not held within the normal range is generated. With this configuration, a subject can more accurately set a position of the sensor 101, and more accurate information can be acquired.

### Eighth Embodiment

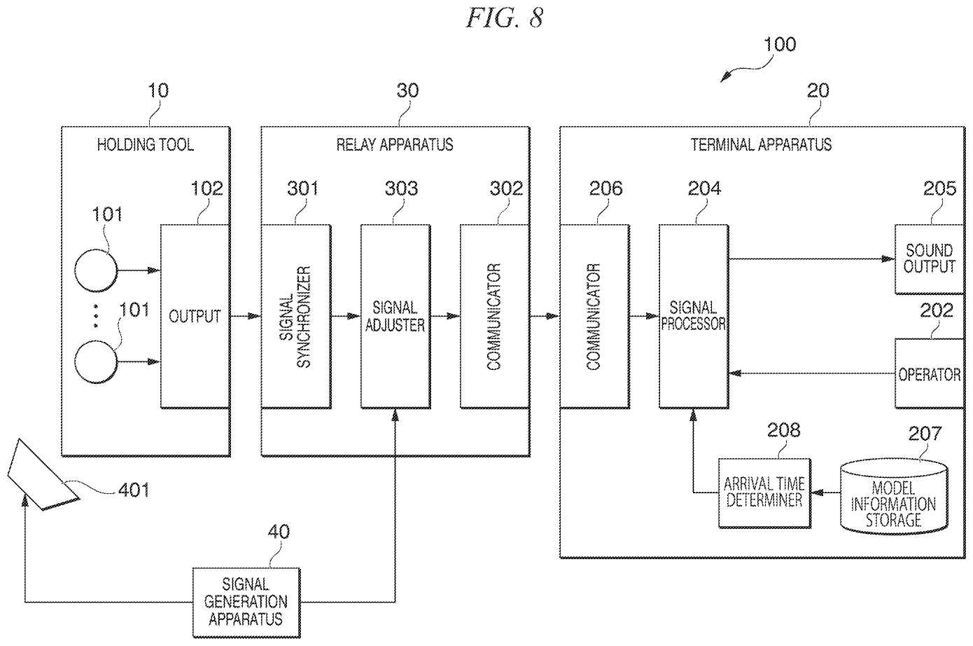

FIG. 8 is a diagram illustrating a system configuration example of an information acquisition system 100 according to an eighth embodiment of the present invention. The information acquisition system 100 according to the eighth embodiment is different from the information acquisition system 100 according to the seventh embodiment in that the terminal apparatus 20 further includes a model information storage 207 and an arrival time determiner 208.

The inside of a living body such as the body of a subject is not filled with a uniform substance, but is composed of many structures having different physical properties. The individual structures have different signal transmission speeds or transmission characteristics. On the other hand, the living body has a common structure to some extent for each species. Therefore, in the eighth embodiment, a structure in a living body of a subject is modeled and used. For example, in a case where auscultation of the chest of the human body is assumed, a skin, subcutaneous tissue, a rib, a bone, a blood vessel, an organ, and the like exist in a predetermined disposition in the chest of the human body, and sound transmission characteristics in each structure can be known. Therefore, information indicating a three-dimensional disposition of the structure is defined in advance as model information and stored in the model information storage 207. The model information storage 207 may store model information for each combination of human generation or gender, for example. A subject in the present invention is not necessarily limited to a human. Therefore, the model information storage 207 may store model information, for example, for each biologically different species (for example, in each of a cat, a dog, a horse, and a cow).

The arrival time determiner 208 uses the model information stored in the model information storage 207 to acquire a signal transmission speed from a certain position of the subject to any position different from the certain position on the basis of a structure present on a path and transmission characteristics thereof (for example, through product-sum calculation). By using the information obtained as described above, the accuracy of signal processing of the signal processor 204 can be improved.

In the information acquisition system 100 according to the eighth embodiment configured as described above, it is possible to perform signal processing with high accuracy in consideration of a structure of a target living body and collect biological information of a target position.

### Ninth Embodiment

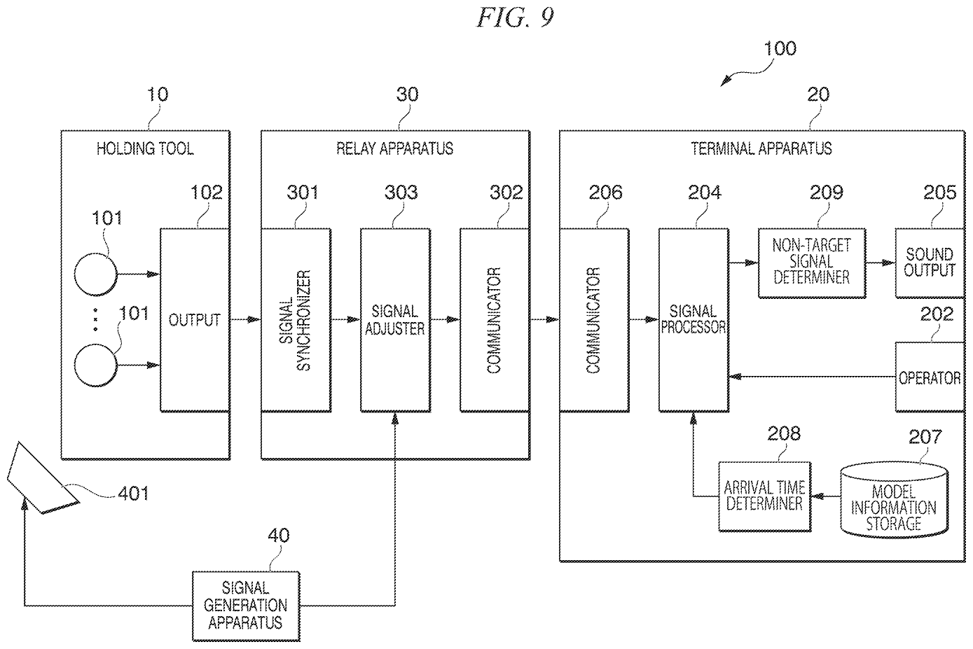

FIG. 9 is a diagram illustrating a system configuration example of an information acquisition system 100 according to a ninth embodiment of the present invention. The information acquisition system 100 according to the ninth embodiment is different from the information acquisition system 100 according to the eighth embodiment in that the terminal apparatus 20 further includes a non-target signal determiner 209.

The non-target signal determiner 209 estimates whether a signal source of a signal that is a processing target is inside or outside a living body of a subject by using the beamforming technique. For example, in a case where a sound signal is a processing target, a sound source position may be estimated on the basis of a signal arrival time. The non-target signal determiner 209 determines that a signal from which a signal source is present outside the living body of the subject is a non-target signal (noise), suppresses this signal component, and outputs the signal. The non-target signal determiner 209 may set a signal satisfying a predetermined condition (for example, a signal of which an amplitude is larger than a predetermined magnitude) as a processing target and determine whether or not the signal is a non-target signal.

In the information acquisition system 100 according to the ninth embodiment configured as described above, it is possible to collect a biological signal at a target position after suppressing a signal (noise) due to the signal source outside the living body of the subject.

### Tenth Embodiment

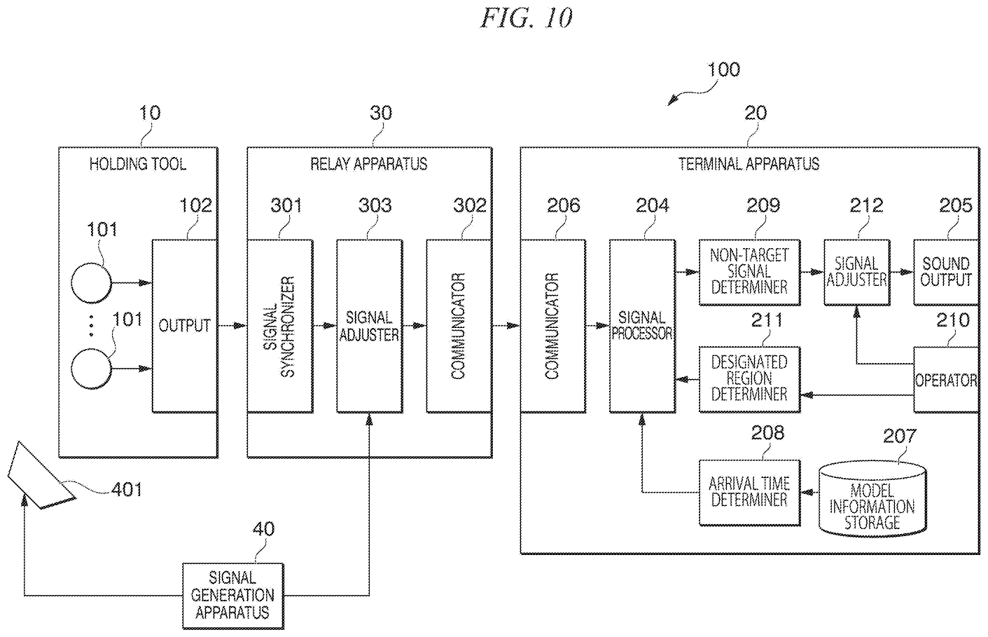

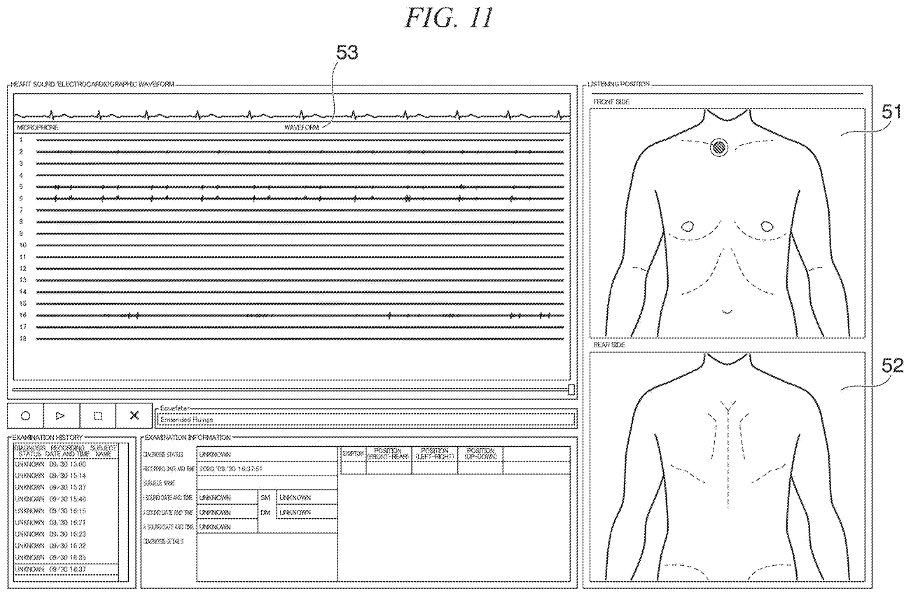

FIG. 10 is a diagram illustrating a system configuration example of an information acquisition system 100 according to a tenth embodiment of the present invention. The information acquisition system 100 according to the tenth embodiment is different from the information acquisition system 100 according to the ninth embodiment in that the terminal apparatus 20 further includes an operator 210, a designated region determiner 211, and a signal adjuster 212.

The operator 210 detects the strength of force (magnitude of pressure) generated at the time of an operation when a user performs an operation (for example, designation of a target position). For example, in a case where a target position is designated through finger pointing on a screen of a tablet terminal, the pressure of the fingertip is detected. The operator 210 outputs the detected pressure to the designated region determiner 211 and the signal adjuster 212.

The designated region determiner 211 receives designation of a region in a depth direction of the living body on the basis of the detected pressure. For example, the designated region determiner 211 may determine that, as the detected pressure becomes larger, a deeper region is designated, and determine that as the detected pressure becomes smaller, a shallower region (a region closer to the surface) is designated. For example, in a case where a target position is designated through finger pointing on a screen of a tablet terminal, it may be difficult to intuitively designate a position in the depth direction because the screen is a two-dimensional plane. On the other hand, the designated region determiner 211 realizes intuitive position designation in the depth direction on the basis of the magnitude of the detected pressure.

The signal adjuster 212 adjusts signal transmission characteristics (for example, frequency characteristics or sound transmission characteristics) on the basis of the magnitude of the detected pressure. In general, in auscultation of the related art, audible sound quality varies depending on the magnitude of the pressure when a chest piece is applied to the body. Therefore, a user applies the chest piece to the body with various pressures to ascertain a disease or a disease state of the subject. Similarly, by changing the sound transmission characteristics such as the frequency characteristics depending on the magnitude of the pressure of the fingertip, it is possible to realize an operational feeling similar to that of a stethoscope of the related art. Change characteristics may be stored in advance by the signal adjuster 212 by measuring a change in the transmission characteristics accompanying a pressure change by using an actual stethoscope.

In the information acquisition system 100 according to the tenth embodiment configured as described above, it is possible to adjust signal transmission characteristics or intuitively designate a three-dimensional target position by detecting a pressure at the time of target position designation.

FIG. 11 is a diagram illustrating a specific example of an operation screen used in the operator (202, 210) of the terminal apparatus 20 in the first to tenth embodiments. In FIG. 11, an image schematically showing the upper half of the human body is displayed on the right side of the operation screen. An image schematically showing the front side of the upper half of the human body is displayed in a first display region 51, and an image schematically showing the rear side of the upper half of the human body is displayed in a second display region 52. The user can designate a target region where a signal is acquired by performing an operation to point to the position of the human body in the first display region 51 or the second display region 52. The acquired information is displayed on the left side of the operation screen. For example, an electrocardiographic waveform and a sound signal waveform may be displayed as illustrated in FIG. 11. By designating a point or a region on the sound signal waveform, the sound signal may be reproduced as sound from the sound output 205. The user may record text indicating the observations. Consequently, other medical practitioners can check the biological information and the observations together.

### Modification Examples

Each apparatus may be divided and mounted in a plurality of casings. For example, the terminal apparatus 20 may be divided and mounted in a plurality of housings. The holding tool 10 and the relay apparatus 30 may be integrally configured. The holding tool 10 and the signal generation apparatus 40 may be integrally configured. The holding tool 10, the relay apparatus 30, and the signal generation apparatus 40 may be integrally configured.

As described above, the embodiments of the present invention have been described in detail with reference to the drawings. On the other hand, the specific configuration is not limited to the embodiments, and includes design and the like without departing from the spirit of the present invention.

### INDUSTRIAL APPLICABILITY

The present invention is applicable to a technique for acquiring biological information.

### REFERENCE SIGNS LIST

100 INFORMATION ACQUISITION SYSTEM
10 HOLDING TOOL
101 SENSOR
102 OUTPUT
111, 112, 113, 114, 20 TERMINAL APPARATUS
201 INPUT
202 OPERATOR
203 OUTPUT
204 SIGNAL PROCESSOR
205 SOUND OUTPUT
206 COMMUNICATOR
207 MODEL INFORMATION STORAGE
208 ARRIVAL TIME DETERMINER
209 NON-TARGET SIGNAL DETERMINER
210 OPERATOR
211 DESIGNATED REGION DETERMINER
212 SIGNAL ADJUSTER
30 RELAY APPARATUS
301 SIGNAL SYNCHRONIZER
302 COMMUNICATOR
303 SIGNAL ADJUSTER
40 SIGNAL GENERATION APPARATUS
401 SIGNAL OUTPUT

1. A terminal apparatus comprising:
an operator that receives information indicating a position in a body of a subject from a user, wherein
biological information corresponding to the position received by the operator is output on the basis of biological information obtained by a plurality of sensors of which positions are substantially fixedly maintained with respect to the body of the subject.

2. The terminal apparatus according to claim 1, wherein
biological information in which biological information obtained by some sensors corresponding to the position received by the operator among the plurality of sensors is emphasized more than biological information obtained by other sensors is output.

3. The terminal apparatus according to claim 1, further comprising:
a signal processor that generates and outputs biological information in which biological information obtained from a direction including the position received by the operator is emphasized more than biological information obtained from another direction on the basis of biological information obtained by the plurality of sensors of which the positions are substantially fixedly maintained with respect to the body of the subject.

4. The terminal apparatus according to claim 1, further comprising:
a signal processor that generates and outputs biological information in which biological information having a region including the position received by the operator as a generation source is emphasized more than biological information having another region as a generation source on the basis of biological information obtained by the plurality of sensors of which the positions are substantially fixedly maintained with respect to the body of the subject.

5. The terminal apparatus according to claim 1, further comprising:
a signal processor that, in a case where a sensor is present in a region including the position received by the operator, estimates and outputs biological information to be captured by the sensor on the basis of biological information obtained by the plurality of sensors of which the positions are substantially fixedly maintained with respect to the body of the subject.

6. An information acquisition system comprising:
a holding tool; and
a terminal apparatus, wherein
the holding tool includes a holding tool main body and a plurality of sensors of which positions are substantially fixedly maintained in the holding tool main body,
the terminal apparatus includes an operator that receives information indicating a position in a body of a subject from a user, and
the terminal apparatus outputs biological information corresponding to the position received by the operator on the basis of biological information obtained by a plurality of sensors of which positions are maintained substantially fixedly with respect to the body of the subject.

7. An information processing method of causing a computer provided with an operator that receives information indicating a position in a body of a subject from a user to execute:
receiving the information indicating the position in accordance with an operation of the user on the operator; and
outputting biological information corresponding to the position received by the operator on the basis of biological information obtained by a plurality of sensors of which positions are maintained substantially fixedly with respect to the body of the subject.
